(19) United States
(12) Patent Application Publication
(10) Pub. No.: US 2026/0240742 A1
(43) Pub. Date: Aug. 20, 2026

(54) UROLITHIN COMBINATION

(71) Applicant: Amazentis SA, Lausanne (CH)

(72) Inventors: Christopher L. Rinsch, Lausanne (CH); Davide D'Amico, Renens (CH); Anurag Singh, Lausanne (CH)

(21) Appl. No.: 19/388,585

(22) Filed: Nov. 13, 2025

(30) Foreign Application Priority Data
Nov. 14, 2024 (GB) 2416802.3

(51) Int. Cl. A61K 8/49; A61Q 19/08

(52) U.S. Cl. CPC A61K 8/498; A61Q 19/08; A61K 2800/592

(57) ABSTRACT

Disclosed are compositions comprising urolithins with active agents, particularly compositions comprising urolithins and one or more retinoid compounds, for example, topical compositions of urolithins with one or more retinoids. Also disclosed are uses of such compositions, for use for the treatment or prevention of diseases, disorders and conditions, particularly diseases, disorder or conditions of the skin and hair, and for use in non-therapeutic treatments. Also disclosed is use of a urolithin, for example, urolithin A, in reducing adverse effects of the administration of a retinoid compound.

### RELATED APPLICATION

This application claims the benefit of priority to UK Patent Application No. GB 2416802.3, filed Nov. 14, 2024.

### BACKGROUND

Healthy mitochondrial function is essential for maintaining skin vitality, resilience, and repair. Mitochondria play a central role in cellular energy production, redox balance, and the regulation of apoptosis—all critical processes for skin homeostasis and regeneration. Impaired mitochondrial function contributes to skin aging, inflammation, and reduced barrier integrity.

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance. See WO2012/088519 (Amazentis SA). In WO2007/127263 (The Regents of the University of California), the use of urolithins for the treatment of various neoplastic diseases is described.

Urolithin A (UA), a gut microbiome-derived metabolite of ellagitannins, has emerged as a potent enhancer of mitochondrial health and mitophagy. UA has demonstrated beneficial effects in models of aging and metabolic dysfunction, but its role in skin health remains underexplored.

International patent publication WO2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, α1-antitrypsin deficiency, ischemia/reperfusion injury, inflammation, aging of the skin, inflammatory bowel disease, Crohn's disease, obesity, metabolic syndrome, type II diabetes mellitus, hyperlipidaemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia (as a result of mitochondrial disease), muscle diseases; cancer, cognitive disorder, stress, and mood disorder.

Retinoids are an umbrella term for a family of natural or synthetic vitamin A derivatives, and they usually have a head-to-tail structure containing four isoprene units (Zasada and Budzisz (2019) Postepy Dermatol Alergol. 36(4):392-397). The term “retinoids” is used to describe compounds that consist of a beta-ionone ring and a polyene side chain with a specific functional group. These functional groups can include an alcohol group, which results in retinol; an aldehyde group, leading to retinal; an acid group, producing retinoic acid (RA); or an ester group, resulting in retinyl ester. Retinol and retinyl ester are types of compounds that are commonly referred to as vitamin A and can be obtained through our diet. However, in order to be biologically active, retinol is converted to retinaldehyde followed by RA by dehydrogenases in a two-step oxidation reaction (Steinhoff et al (2022) Nutrients. 14(6):1236). RA exists in different forms, with the two most prevalent forms being all-trans RA (ATRA) and 9-cis RA. RA itself is rapidly metabolized by cytochrome P450 (CYPs), resulting the hydroxylation of ATRA and inactivation of its functions (Ghyselinck and Duester (2019) Development. 146(13):dev167502).

Based on their molecular and structural properties, retinoids can be divided into four generations. First-generation retinoids, such as retinol and its metabolites, are either naturally occurring or non-aromatic compounds modified from the polyene vitamin. They do not act selectively towards retinoid receptors. Second-generation retinoids are synthetic compounds that are either monoaromatic retinoids, where the cyclohexene ring is replaced by a benzene ring, or synthetic analogues of vitamin A. Third-generation retinoids with cyclic polyene side-chain are recognized by selective activity towards RA receptors-β (RAR-β) and RA receptors-γ (RAR-γ) receptors. The newly developed fourth generation of retinoids are characterized by binding selectively to RAR-γ pre-dominantly in epidermis.

Retinoids play important roles in regulating a wide range of biological processes, including cell proliferation, differentiation, apoptosis, inflammation and development. Therefore, they have also been demonstrated in clinical pharmacotherapy for the treatment of vision-related disorders, skin-related disorders, and cancer. Retinol has also been widely used in cosmetic formulations to reduce wrinkles and improve cellulite and has been approved by the Food and Drug Administration (FDA) for anti-aging treatments since 1996. The anti-aging and anti-photoaging effects of retinoids are based on the promotion of keratinocyte proliferation collagen and elastin fibre synthesis, and the inhibition of collagen degradation by inhibiting metalloproteinase activity. These effects ultimately result in a deceleration of the aging process, enhancement of repair mechanisms, and reduction of wrinkles and hyperpigmentation.

Nevertheless, the vast cosmetic and clinical applications of retinoids have been limited by three main obstacles. Firstly, poor water-solubility, secondly, photosensitivity and thirdly side effects, including severe local irritation in the form of a burning sensation, itching, erythema, peeling, or dryness. These properties limit their therapeutic efficacy.

### SUMMARY

The objective of the present invention was to identify molecules capable of reducing the discomfort, irritation, and inflammatory responses induced by retinol, while preserving or enhancing its beneficial effects on skin health. Specifically, the invention aims to leverage the mitochondrial-protective properties of urolithins, such as urolithin A to counterbalance the irritation induced by retinoid compounds, such as retinol, by activation of PINK1 mitophagy pathways and by reducing detrimental inflammation. PINK1 (PTEN-induced kinase 1) is a key regulator of mitophagy, the selective degradation of damaged mitochondria. Upregulation of PINK1 promotes mitochondrial quality control, thereby supporting cellular health and resilience, particularly in tissues with high metabolic demand such as the skin.

The invention relates to compositions comprising urolithins with active agents, particularly compositions comprising urolithins and one or more retinoid compounds, for example, topical compositions of urolithins with one or more retinoids. The invention also relates to uses of such compositions, for use for the treatment or prevention of diseases, disorders and conditions, particularly diseases, disorder or conditions of the skin and hair, and for use in non-therapeutic treatments. The invention also related to a urolithin, for example, urolithin A, for use in reducing adverse effects of the administration of a retinoid compound.

Therefore, according to the first embodiment of the invention there is provided a composition comprising:
(a) a compound of formula (I), or a salt, prodrug, metabolite or derivative thereof;

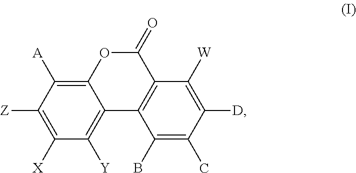

wherein:
A, B, C, D, W, X, Y and Z are each independently selected from H and OH; and
(b) one or more retinoid compounds.

In a further embodiment, there is provided a compound of formula (I), for example, urolithin A, for use in reducing adverse effects of the administration of a retinoid compound.

In one embodiment, the retinoid compound is a first-generation retinoid, for example, a retinoid compound selected from one or more of retinol, retinal, tretinoin (all trans retinoic acid), isotretinoin (13 cis-retinoic acid), and alitretinoin (9 cis-retinoic acid). Retinol also comprises esters of retinol, such as retinol acetate, retinol palmitate, and retinol propionate.

In a further embodiment, the retinoid compound is a second-generation retinoid, for example, a retinoid compound selected from one or more of etretinate and its metabolite acitretin.

In a further embodiment, the retinoid compound is a third-generation retinoid, for example, a retinoid compound selected from one or more of adapalene, bexarotene, etarotene, tazarotene, motretinide, arotinoide and arotinoidethylester.

In a further embodiment, the retinoid compound is a fourth-generation retinoid, for example, trifarotene.

In a further embodiment, the retinoid compound is selected from retinol (Vitamin A), retinal (retinaldehyde), retiferol, tretinoin (retinoic acid), isotretinoin, alitretinoin (9-cis-retinoic acid), etretinate, acitretin, adapalene, bexarotene, tazarotene or trifarotene.

In a further embodiment, the retinol is a retinol isomer, for example, all-trans-retinol, 13-cis-retinol, 11-cis-retinol, 9-cis-retinol, 3,4-didehydro-retinol, 3,4-didehydro-13-cis-retinol; 3,4-didehydro-11-cis-retinol; or 3,4-didehydro-9-cis-retinol.

In a further embodiment, the retinoid compound is selected from Seletinoid G, AGN-193109, AGN-190168, AGN-190299 and rexinoid analogues such as, LGD1069 and LG100268.

In a further embodiment, the retinoid compound is selected from:
(a) retinoid hybrids, for example, retinyl retinoate, bis-retinamido methylpentane and retinyl gallate;
(b) soft retinoids, such as hydroxypinacolone retinoate; and
(c) retinoid mimetics/non-acidic analogues, such as bakuchiol, azelaic acid, Vigna aconitifolia Extract and phytol.

In a further embodiment, the retinoid is a compound comprising a retinoid structure, a retinoid metabolite, or an agent that can be metabolized into a retinoid or retinoid metabolite. A retinoid further includes a compound that is an analogue or mimic of a retinoid or a retinoid metabolite, or an agent that can be metabolized into an analogue or mimic of a retinoid or a retinoid metabolite.

In a further embodiment the retinoid can be any retinoid disclosed in U.S. Pat. Nos. 5,648,563; 5,648,385; 5,618,839; 5,559,248; 5,616,712; 5,616,597; 5,602,135; 5,599,819; 5,556,996; 5,534,516; 5,516,904; 5,498,755; 5,470,999; 5,468,879; 5,455,265; 5,451,605; 5,426,118; 5,407,937; 5,399,586; 5,399,561; 5,391,753. [0045]

In a further embodiment, the retinoid compound is a retinoic acid.

In a further embodiment, the retinoid is all-trans retinoic acid (ATRA).

In a further embodiment, a composition disclosed herein for use in the treatment or prevention of a skin disease, disorder, or condition, for example acne, dermatitis, eczema, hives, liver spots (lentigo), lupus erythematosus, hyperpigmentation, keratosis pilaris, melasma, psoriasis, rosacea, shingles or warts, for example, wherein the acne is selected from acne vulgaris, nodulocystic acne, acne conglobate, skin infection, skin irritation, skin inflammation or skin damage caused by radiation, for example, UV, beta or gamma radiation, for example, during medical treatment for a condition, such as a cancer, such as cutaneous T-cell lymphoma, and Kaposi's sarcoma.

In a further embodiment, a composition disclosed herein a non-therapeutic use for: enhancing skin health in a generally healthy subject; educing, slowing or preventing skin aging, and/or lowering skin biological age; reducing or preventing skin wrinkles and/or maintaining or improving skin barrier function; maintaining or improving skin hydration and/or skin elasticity; reducing irradiation-induced skin damage, for example, damage caused by UV radiation; improving or boosting skin collagen levels; supporting collagen function and/or architecture; preventing collagen decline; supporting skin collagen levels; and/or supporting skin collagen levels; and/or maintaining or enhancing skin collagen health.

### BRIEF DESCRIPTION OF THE FIGURES

FIG. 1: Bar graph showing gene expression of MMP1 in HDF cells following treatment with UA 2.5 uM, retinoic acid 2.5 uM or combination of the two for 24 h. DMSO used as control. Data represent relative fold change expression compared to DMSO and are indicated as mean+/−SEM. Statistical test One Way ANOVA with Tukey multiple comparisons.

FIG. 2: Bar graph showing gene expression of MMP1 in HDF cells following treatment with UA 10 uM, retinoic acid 10 uM or combination of the two for 24 h. DMSO used as control. Data represent relative fold change expression compared to DMSO and are indicated as mean+/−SEM. Statistical test One Way ANOVA with Tukey multiple comparisons.

FIG. 3: Effect of retinol and urolithin A treatments on PINK1 mRNA expression in human keratinocyte cells (NHEK). Relative fold change in PINK1 expression was quantified by qPCR and normalized to vehicle control. Data represent mean±standard deviation. Treatments include Urolithin A at 10 μM alone, retinol at 10 μM alone and combined with urolithin A at 10 μM. Statistical significance was determined using one-way ANOVA; ***p<0.001.

FIG. 4: Effect of retinol and urolithin A treatments on IL1α mRNA expression in NHEK cells. Relative fold change in IL1α expression was quantified by qPCR and normalized to vehicle control. Data represent mean±standard deviation. Treatments include retinol at 10 μM alone and combined with urolithin A at 2.5 μM or 10 μM. Statistical significance was determined using one-way ANOVA; ***p<0.001, ****p<0.0001.

### DETAILED DESCRIPTION

### Compounds of Formula (I) (Urolithins)

Compounds of formula (I) (Urolithins) are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in Table 1 below, with reference to structure (I).

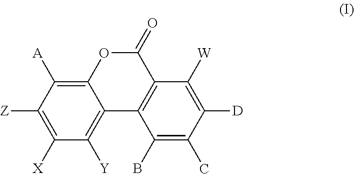

| | Substituent of structure (I) | | | | | |
|---|---|---|---|---|---|---|
| | | | | | W, X | |
| | A | B | C | D | and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972, all of which are incorporated by reference.

Urolithins of any structure according to structure (I) may be used in the combinations of the invention.

In one aspect of a combinations of the invention, a suitable compound is a compound of formula (I) wherein A, C, D and Z are independently selected from H and OH and B, W, X and Y are all H, preferably at least one of A, C, D and Z is OH.

Particularly suitable compounds are the naturally occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

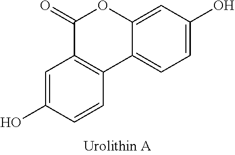 [SMILES: O=c1oc2cc(O)ccc2c2ccc(O)cc12]

According to one embodiment there is provided a combination, composition, use or method of the invention wherein the compound of formula (I) is urolithin A.

According to one embodiment there is provided a combination, composition, use or method of the invention wherein the compound of formula (I) is urolithin B.

According to one embodiment there is provided a combination, composition, use or method of the invention wherein the compound of formula (I) is urolithin C.

According to one embodiment there is provided a combination, composition, use or method of the invention wherein the compound of formula (I) is urolithin D.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucamine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

### Urolithin Administration/Dosage Regimes

The administration of compositions of the present invention preferably involves topical administration of a urolithin of formula (I) or salt thereof to a subject. In one embodiment, concentrations of urolithins are in the range of about 0.25% (w/w) to about 5% (w/w) of the composition, such as about 0.5% (w/w) to about 4% (w/w), for example. About 0.5% (w/w) to about 3% (w/w), or about 0.5% (w/w) to about 2% (w/w) or about 1% (w/w) to about 2% (w/w), for example, about 1% (w/w), about 1.5% (w/w) or about 2% (w/w).

In an alternative embodiment the administration of compositions of the present invention involves oral administration of a urolithin of formula (I) or salt thereof to a subject, for example, in a daily amount in the range of 1.7 to 6.0 mmol per day, for example, from about 1.7 to about 2.7 mmol per day, or from about 2.8 to about 6.0 mmol per day. In a further embodiment, oral administration is in the range 125 mg to 2000 mg urolithin A or 250 mg to 2000 mg urolithin A (which corresponds to about 1.1 to 8.8 mmol), for example 125 mg to 1500 mg or 250 mg to 1500 mg, such as 125 mg to 1000 mg or 250 mg to 1000 mg. In one embodiment the oral dose is 125 mg/day or 250 mg/day, in an alternative embodiment the oral dose is 500 mg/day and in another embodiment the oral dose is 1000 mg/day. In a further embodiment, the oral dose is 1500 mg/day. In a further embodiment, the oral dose is 2000 mg/day.

In a further embodiment, oral administration doses are selected from:
125 mg once or twice per day
250 mg once or twice a day;
500 mg once or twice a day;
750 mg once or twice a day;
1000 mg once or twice a day;
1250 mg once or twice a day; or
1500 mg once or twice a day

When dosed orally, the uses and methods of the present invention involve daily administration of the compound of formula (I) or salt thereof, or of a composition comprising the compound or salt. In some embodiments, the compound or composition is administered once per day, i.e. the compound or composition is to be administered at least once per 24 hour period. In other embodiments the compound, or composition comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

When dosed orally, the methods of the present disclosure would usually require daily administration of the compound of formula (I) or salt thereof, or of a composition containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the compound or salt thereof daily for a period of up to 3 months, up to 6 months, up to 1 year, up to 2 years or up to 5 years. In some embodiments, the method comprises administering the compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

When dosed orally, the uses or methods of the present disclosure require daily administration of an amount of compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4. 1.5, 1.6 1.7, 1.8, 1.9, 2.0, 2.1, 2.2, 2.3, 2.4, 2.5, 2.6, or 2.7 mmol. In some preferred embodiments the uses or method involves administration of approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

When dosed orally, in a further embodiment, the methods of the present disclosure require daily administration of an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the use or method involves administration of approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

When dosed orally, in some embodiments the methods involve administration of urolithin A in an amount in the range of from 400 to 600 mg/day or 400 to 600 mg twice per day. In a preferred embodiment the method involves administration of urolithin A in an amount in the range of from 450 to 550 mg, more preferably approximately 500 mg per day or twice per day.

When dosed orally, in other embodiments the methods involve administration of urolithin A in an amount in the range of from 700 to 1300 mg/day twice per day, or in the range of from 750 to 1250 mg, or in the range of from 800 to 1200 mg, or in the range of from 850 to 1150 mg, or in the range of from 900 to 1100 mg per day or twice per day. In a preferred embodiment the method involves administration of urolithin A in an amount in the range of from 950 to 1150 mg/day or twice per day, more preferably approximately 1000 mg/day or twice per day.

When dosed orally, in some preferred embodiments, the uses or methods involve administering urolithin A to the subject in an amount in the range of from 4.5 to 11 mg/kg/day, such as 4.5 to 8.5 mg/kg/day. In another embodiment, the uses or methods involve administering urolithin A to the subject in an amount in the range of 5 to 9 mg/kg/day. In another embodiment, the uses or methods involve administering urolithin A to the subject in an amount in the range of from 6.0 to 8 mg/kg/day.

When dosed orally, in other preferred embodiments, the uses or methods involve administering urolithin A to the subject in an amount in the range of from 9 to 18 mg/kg/day such as 9 to 17 mg/kg/day. In another embodiment, the uses or methods involve administering urolithin A to the subject in an amount in the range of from 10 to 17 mg/kg/day. In another embodiment, the uses or methods involve administering urolithin A to the subject in an amount in the range of from 11 to 16 mg/kg/day.

When dosed orally dosage regimes which combine a 500 mg dose and a 1000 mg dose may be advantageous. For example, a twice daily dosage regime which combines a first dose of 1000 mg and a second dose several hours later of 500 mg. Said 500 mg dose may be 6-18 hours after the 1000 mg dose, for example 8-12 hours after the 1000 mg dose. For example, about 12 hours after the 1000 mg dose. Thus, according to a further aspect of the invention there is provided the treatment of a disease, disorder or condition with a compound of Formula (I) which comprises a twice daily dosage regime comprising a first dose of 1000 mg, followed by a second dose of 500 mg wherein the two doses are separated by 6-18 hours.

The compound of formula (I) or salt thereof, or composition containing the compound of salt, may be administered at any suitable time, for example, it may be administered in the morning after sleep or in the evening. In some embodiments, it may be preferable for the method to be performed at approximately the same time(s) each day, for example within 15, 30, 60 or 120 minutes of a given time point.

In a further embodiment, there is provided an oral composition wherein the compound of formula (I) is administered at a dose of about 4.5-18 mg/kg.

### Retinoid Administration/Dosage Regimes

The administration of compositions of the present invention preferably involves topical administration of a retinoid or salt thereof to a subject. In one embodiment, concentrations of urolithins are in the range of about 0.05% (w/w) to about 1% (w/w) of the composition, such as about 0.1% (w/w) to about 1% (w/w), for example. About 0.2% (w/w) to about 0.8% (w/w), or about 0.3% (w/w) to about 0.7% (w/w) or about 0.4% (w/w) to about 0.6% (w/w), for example, about 0.1% (w/w), about 0.3% (w/w), about 0.5% (w/w), 0.7% (w/w) or about 1% (w/w).

In a further embodiment, topical administration of compositions comprising more potent retinoids, such as tretinoin, are in the range about 0.005% (w/w) to about 0.2% (w/w) of the composition, such as 0.01% (w/w) to about 0.1% (w/w), for example, about 0.02% (w/w) to about 0.08% (w/w), or about 0.03% (w/w) to about 0.07% (w/w) or about 0.04% (w/w) to about 0.06% (w/w), for example, about 0.01% (w/w), about 0.03% (w/w), about 0.05% (w/w), 0.07% (w/w) or about 0.1% (w/w).

In a further embodiment, a suitable, non-limiting example of a dosage of a retinoid or pharmaceutically acceptable salt thereof according to the present invention or a composition comprising such a retinoid or pharmaceutically acceptable salt thereof, is from about 1 ng/kg to about 1000 mg/kg, such as from about 1 mg/kg to about 100 mg/kg, including from about 5 mg/kg to about 50 mg/kg. Other representative dosages of a compound or a composition of the present invention include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

In certain embodiment, the retinoid or pharmaceutically acceptable salt thereof, is administered at a dose of about 2 mg/kg, 5 mg/kg, 10 mg/kg, or 20 mg/kg.

In certain embodiments, the retinoid or pharmaceutically acceptable salt thereof may be administered hourly, daily, weekly, monthly, yearly or as a one-time delivery. In a further embodiment, the retinoid or pharmaceutically acceptable salt is administered for one 1, 2, 3, 4, 5, 6, 7, 8, 9, 10, 11, 12, 13, 14, 15 or up to 16 wks. In one embodiment, the retinoid is administered every other day. In a further embodiment, the retinoid is administered once per day, every day. In a further embodiment, the retinoid is administered twice per day, every day.

In a further embodiment the compositions of the invention are administered topically.

In a further embodiment the compositions of the invention are administered orally.

Combinations of the invention may be administered in a mixture of oral and topical formulations, wherein one dosage form (either topical or oral) contains a urolithin, for example, urolithin A, and the other dosage form (either topical or oral) comprises a retinoid compound. For example, wherein the topical formulation comprises a retinoid compound and the oral formulation comprises a urolithin, for example, urolithin A. For example, wherein the topical formulation does not include a urolithin. For example, wherein that topical formulation contains a combination of a retinoid compound and a urolithin, for example, urolithin A.

### Additional/Combination Therapy

Compositions of the invention may comprise one or more further active agents, for use in the treatment or prevention of diseases, disorders or conditions. Examples of such further active ingredients, include one or more of the following compounds: hyaluronic acid, niacinamide, vitamin C, E and/or glutathione.

Any active agent which is known to be useful, or which has been used or is currently being used for the treatment or prevention of diseases, disorders or conditions can be used with a combination of the invention. See, e.g., Gilman et al, Goodman and Gilman's: The Pharmacological Basis of Therapeutics, 13th ed., McGraw-Hill, New York, 2017; The Merck Manual of Diagnosis and Therapy, Robert S. Porter, M. D. et al. (eds.), 20th Ed., Merck Sharp & Dohme Research Laboratories, Rahway, NJ, 2018; Cecil Textbook of Medicine, 25th Ed., Goldman and Schafer (eds.), Elsevier, 2015, and Physicians' Desk Reference (71st ed. 2016) for information regarding therapies (e.g., prophylactic or therapeutic agents) which have been or are currently being used for the treatment or prevention of diseases, disorders or conditions associated with hair, nails and/or skin.

### Uses

Compositions of the invention can be used for both therapeutic and non-therapeutic uses, finding use in the treatment of various diseases as well as health conditions not considered to be a disease. In particular, disease and non-disease health conditions may be characterised by an inadequate mitochondrial activity. Therefore, according to one embodiment of the invention there is provided a composition of the invention for use as a medicament for the treatment of a disease disorder or condition. According to a further embodiment of the invention there is provided a non-therapeutic method of use of a composition of the invention.

The compositions of the invention include any suitable topical composition, for example in the form of a topical cream, moisturizer, serum or oil.

Compositions of the invention find utility in the treatment of disease, disorders and conditions. Therefore, according to a further aspect of the invention, there is provided a composition, comprising:
(a) a compound of formula (I), or a salt, prodrug, metabolite or derivative thereof; and
(b) one or more retinoid compounds;
for use as a medicament for the treatment of a disease disorder or condition.

According to a further aspect of the invention there is provided a composition, comprising
(a) a compound of formula (I), or a salt, prodrug, metabolite or derivative thereof, for example, urolithin A; and
(b) one or more retinoid compounds;
for use in the treatment, prevention or management of a mitochondria-related condition associated with altered mitochondrial function or reduced mitochondrial density.

According to a further aspect of the invention there is provided a non-therapeutic method for preventing or managing a mitochondria-related condition associated with altered mitochondrial function or reduced mitochondrial density, comprising administration of an effect amount of a composition comprising:
(a) a compound of formula (I), or a salt, prodrug, metabolite or derivative thereof, for example, urolithin A; and
(b) one or more retinoid compounds;

According to a further aspect of the invention there is provided a combination or composition, comprising
(a) a compound of formula (I), or a salt, prodrug, metabolite or derivative thereof, for example, urolithin A; and
(b) one or more retinoid compounds;
for use in the treatment, prevention or management of a mitochondria-related disease associated with altered mitochondrial function or reduced mitochondrial density.

According to a further aspect of the invention there is provided a composition, comprising
(a) a compound of formula (I), or a salt, prodrug, metabolite or derivative thereof, wherein the compound of formula (I) is a urolithin, for example, urolithin A, urolithin B, urolithin C or urolithin D, such as urolithin A; and;
(b) one or more retinoid compounds;
for use in the treatment, prevention or management of a mitochondria-related disease.

According to a further aspect of the invention there is provided a combination or composition, comprising
(a) a compound of formula (I), or a salt, prodrug, metabolite or derivative thereof, wherein the compound of formula (I) is a urolithin, for example, urolithin A, urolithin B, urolithin C or urolithin D, such as urolithin A; and;
(b) one or more retinoid compounds;
for use in increasing or maintaining mitochondrial function.

According to a further aspect of the invention there is provided a non-therapeutic method of increasing or maintaining mitochondrial function, comprising administration of an effect amount of a composition comprising:
(a) a compound of formula (I), wherein the compound of formula (I) is a urolithin, for example, urolithin A, urolithin B, urolithin C or urolithin D, such as urolithin A; and
(b) one or more retinoid compounds.

### Therapeutic Uses

### Skin Diseases, Disorders and Conditions

According to one embodiment of the invention, there is provided a composition of the invention for use in the treatment or prevention of a skin disease, disorder or condition. In one embodiment, the skin disease, disorder or condition is selected from acne. psoriasis, skin cancers (for example, cutaneous T-cell lymphoma, and Kaposi's sarcoma), keratosis pilaris and hyperpigmentation.

In a further embodiment the invention provides a composition of the invention for the treatment or prevention of a skin disease, disorder or condition wherein the disease, disorder or condition is selected from acne, dermatitis, eczema, hives, liver spots (lentigo), lupus erythematosus, hyperpigmentation, keratosis pilaris, melasma, psoriasis, rosacea, shingles or warts, for example, wherein the acne is selected from acne vulgaris, nodulocystic acne, and acne conglobate.

In a further embodiment the invention provides a composition of the invention for the treatment or prevention of a skin disease, disorder or condition wherein the disease, disorder or condition is selected from skin infection, skin irritation, skin inflammation or skin damage caused by radiation, for example, UV, beta or gamma radiation, for example, during medical treatment for a condition, such as a cancer.

In a further embodiment the invention provides a composition of the invention for the treatment or prevention of a skin disease, disorder or condition wherein the disease, disorder or condition is cancer, for example, cutaneous T-cell lymphoma, and Kaposi's sarcoma.

In a further embodiment, acne is selected from acne vulgaris, nodulocystic acne, and acne conglobate.

In a further embodiment of the invention, there is provided a composition of the invention for use in the treatment of one or more indications, selected from:
(a) acne;
(b) contact dermatitis
(c) eczema;
(d) psoriasis;
(e) rosacea,
(f) lupus erythematosus;
(g) warts;
(h) shingles;
(i) hives;
(j) melasma;
(k) liver spots (lentigo);
(l) skin irritation;
(m) skin infection;
(n) skin inflammation;
(o) seborrheic dermatitis;
(p) Skin fungal infections (for example, candidiasis, athletes' feet);
(q) Skin cancer;
(r) diaper rash;
(s) for protecting skin from damage caused by radiation, e.g. UV, beta or gamma radiation including during medical treatment for a condition, such as a cancer; and
(t) for reduction of skin damage by irradiation, for example UV irradiation-induced skin damage.

### Aging/Healthspan

According to a further embodiment of the invention, there is provided a composition of the invention for use in the increasing or prolonging of skin and/or hair healthspan. Healthspan can be defined as the part of a person's life during which the skin and/or hair are generally in good health. In a further embodiment of the invention, there is provided a composition of the invention, for enhancing skin and/or hair longevity. In a further embodiment of the invention, there is provided a composition of the invention for decreasing or slowing skin and/or hair deterioration due to aging.

### Treatments

The composition of the invention can be taken as a single treatment or, more commonly, as a series of treatments. In one example, a subject takes a dose before or after exercise. For a subject who is not able to exercise, a dose of the composition may, for example, be taken once, twice or three times per day, or one, two, three, four, five or six times per week. In another example, the intervention may be taken by a subject independent of the subject's ability or need to exercise. It will also be appreciated that the effective dosage of the compound may increase or decrease over the course of a particular treatment.

### Non-Therapeutic Uses

### Skin

In a further embodiment, there is provided a non-therapeutic method for the treatment of skin conditions comprising the administration of a composition of the invention. In a further embodiment of the invention, there is provided a method for:
(a) renewal and/or revitalization of skin appearance;
(b) improvement in skin texture;
(c) enhancement of skin radiance;
(d) skin rejuvenation;
(e) reduction of pore and micro line visibility;
(f) improvement in skin respiration.
(g) support optimal cellular function in skin;
(h) promote/activate skin detoxifying processes;
(i) promote collagen repair, and/or
(j) improve skin stem cell health
(k) improving or maintaining skin barrier function;
(l) for limiting trans-epidermal water loss;
(m) for improving skin elasticity and firmness;
(n) for increasing or maintaining skin layer thickness;
(o) for increasing or maintaining the undulation index of dermal epidermal junctions in skin;
(p) for increasing or maintaining skin hydration;
(q) for reducing skin biological aging;
(r) for diminishing fine lines and wrinkles;
(s) for improving collagen organization in the skin;
(t) for improving skin matrix;
(u) improving skin physical properties;
(v) decreasing sun-induced skin redness; or
(w) for protection from sun-induced photoaging;
comprising the administration of a composition of the invention.

### Hair

In a further embodiment of the invention, there is provided a composition of the invention for example, a composition comprising urolithin A, for one or more of the following uses:
a) for stimulating hair growth;
b) the treatment of hair loss;
c) preventing or ameliorating hair loss;
d) for delaying the onset of hair loss;
e) for treating hair thinning;
f) increasing hair thickness (i.e. number of hair fibres per surface area) and/or number hair follicles actively producing hair fibres; and/or
g) slowing or preventing premature greying of hair.

In a further embodiment of the invention, there is provided a composition of the invention for example, a composition comprising urolithin A, for one or more of the following uses:
a) maintaining or enhancing hair thickness;
b) hair follicle cell regeneration;
c) hair follicle cell survival;
d) hair stem cell growth and/or regeneration;
e) hair cell survival;
f) hair loss prevention,
g) promoting new hair growth;
h) scalp health improvement,
i) for improving survival of hair transplants; and/or
j) hair growth restoration promotion;
k) for reduction of hair damage by irradiation, for example UV irradiation-induced hair damage;
l) for protecting hair from damage caused by radiation, e.g. UV, beta or gamma radiation including during medical treatment for a condition such as a cancer.

In a further embodiment of the invention, there is provided a composition of the invention for example, a composition comprising urolithin A, for one or more of the following uses:
a) enhancing hair stem cell function;
b) enhancing hair follicle elongation,
c) enhancing hair matrix proliferation, for example, as measured by Ki67 levels;
d) enhancing hair growing phase, (enhancing the duration of the anaphase);
e) hair matrix proliferation,
f) inhibition of hair matrix apoptosis; and
g) reducing melanin clumping, for example, in pigmentary units, for example, in hair follicle pigmentary units.

### Compositions

The uses and methods of the present invention preferably involve topical administration. The compositions may be made into a wide variety of articles that include but are not limited to lotions, creams, gels, sticks, sprays, ointments, cleansing liquid washes and solid bars, shampoos and hair conditioners, pastes, foams, powders, mousses, shaving creams, wipes, strips, patches, electrically-powered patches, wound dressing and adhesive bandages, hydrogels, film-forming products, facial and skin masks, make-up such as foundations, eye liners, and eye shadows, and the like. These product types may contain several types of cosmetically-acceptable carriers including, but not limited to solutions, suspensions, emulsions such as microemulsions and nanoemulsions, gels, solids and liposomes. Such articles may be distributed as a pharmaceutical, an over-the-counter medication, or cosmetic. The following are non-limitative examples of such carriers. Other carriers can be formulated by those of ordinary skill in the art.

The compositions useful in the present invention can be formulated as solutions. Solutions typically include an aqueous or organic solvent (e.g., from about 50% to about 99.99% or from about 90% to about 99% of a cosmetically acceptable aqueous or organic solvent). Examples of suitable organic solvents include but are not limited to ethanol, glycols (such as butylene glycol), glycerine, ethoxydiglycol, and dimethyl isosorbide.

According to a further embodiment of the invention, there is provided a topical composition, comprising:
(a) a compound of formula (I); or a salt thereof, and/or one or more retinoid compounds
(b) one or more of the following:
   i) an aqueous phase;
   ii) a water soluble emollient and humectant;
   iii) a co-solvent/wetting agent
   iv) an occlusive moisturizer and barrier repair ingredient, such as a vitamin rich occlusive moisturizer and barrier repair ingredient;
   v) a carrier oil/emollient, such as a C₈₋₁₄ medium chain mono-ester;
   vi) an emulsifier;
   vii) a structuring agent;
   viii) a polymeric thickener and emulsifier; for example, a C₁₅₋₁₉ alkane,
   ix) a polymeric anionic thickener; and/or
   x) a preservative.

According to a further embodiment of the invention, there is provided a topical composition, comprising:
(a) a compound of formula (I), or a salt thereof, and/or one or more retinoid compounds;
(b) a carrier oil/emollient, such as a C₈₋₁₄ medium chain mono-ester; and
(c) an emulsifier.

According to a further embodiment of the invention, there is provided a topical composition, comprising:
(a) a compound of formula (I), or a salt thereof, and/or one or more retinoid compounds;
(b) a water soluble emollient and humectant;
(b) a carrier oil/emollient, such as a C₈₋₁₄ medium chain mono-ester; and
(c) an emulsifier.

According to a further embodiment of the invention, there is provided a topical composition, comprising:
(a) a compound of formula (I), or a salt thereof, and/or one or more retinoid compounds;
(b) a water soluble emollient and humectant;
(c) a carrier oil/emollient, such as a C₈₋₁₄ medium chain mono-ester;
(d) an emulsifier
(e) a structuring agent; and
(f) a polymeric thickener.

Examples of water soluble emollient and humectants include: glycerine, butylene glycol, 1,2-hexanediol

Examples of co-solvent/wetting agents include: ethanol, glycols (such as butylene glycol), glycerin, ethoxydiglycol, dimethyl isosorbide.

Examples of occlusive moisturizer and barrier repair ingredients include butyrospermum parkii (shea) butter;

Examples of carrier oil/emollients include: coco-caprylate/caprate, such as mono-, di-, and tri-esters of spanning the range of C₁ to C₂₂. Fats and oils (defined as triesters of glycerol), natural or synthetic. Lamellar forming structures such as those mentioned above, cholesterol, phytosterol.

Examples of emulsifiers include; sorbitan oleate, PEG-100 stearate,

Examples of structuring agents include; stearic acid

Examples of polymeric thickener and emulsifiers include: hydroxyethyl acrylate/sodium acryloyldimethyl taurate copolymer,

Examples of polymeric anionic thickeners include: carbomer

Examples of preservatives include: phenoxyethanol.

Examples of surfactants include C₈ to C₂₂ ionic or non-ionic. The more water soluble ones (higher Hydrophile Lipophile Balance or HLB) tend to disrupt the skin barrier and allow substances to transverse more readily.

In an alternative embodiment, the uses and methods of the present invention involve oral administration of the compositions of the invention. Any suitable oral composition may be used. Accordingly, the use of a range of compositions which are suitable for oral administration, is envisaged. Thus, in some embodiments, the composition is administered in the form of an oral composition and one or more excipients suitable for oral administration. Oral compositions may comprise compositions having the form of a pill, tablet, capsule, caplet, lozenge, pastille, granules, powder for suspension, oral solution, oral suspension, oral emulsion, syrup, or the like.

Compositions may take any physical form suitable for the intended application, for example, they may be in the form of a solid (for example, a tablet or capsule), a semi-solid (for example, a softgel), or a liquid (including emulsions). In some instances, the composition may be in the form of a viscous fluid or a paste. Semi-solid forms may likewise contain excipients conventional in the art. The excipients can, for example, provide a desired hardness, shelf-life and flavour such that the composition has an acceptable taste, an attractive appearance and good storage stability. Semi-solid forms can be in the form of a paste. Where the composition is a softgel, it may for example be provided in a capsule having a shell. The shell may be of a conventional type, for example it may be a soft gelatin-based shell. By way of example, the composition may also be provided inside a hard capsule type of shell. Liquid compositions may be in the form of a medicine, a dietary supplement, or a beverage, each for oral consumption. Liquid formulations may be solutions, emulsions, slurries or other semi-liquids. Excipients in a liquid composition can, for example, provide a shelf-life, visual appearance, flavour and mouth-feel such that the composition has an acceptable taste, an attractive appearance and good storage stability. At certain levels of dilution, a drink may need to be shaken before the subject drinks it, so as to maintain an even suspension of the active ingredient.

In some preferred embodiments, the use or method comprises administration of the compound of formula (I) or salt thereof (e.g. urolithin A), in micronized form. Micronization enables the compound of formula (I) to disperse or dissolve more rapidly. Micronisation can be achieved by methods established in the art, for example compressive force milling, hamermilling, universal or pin milling, or jet milling (for example spiral jet milling or fluidised-bed jet milling) may be used. Jet milling is especially suitable. If micronized compound is used, then preferably the compound has a D₅₀ size of under 100 μm—that is to say that 50% of the compound by mass has a particle diameter size of under 100 μm. More preferably, the compound has a D₅₀ size of under 75 μm, for example under 50 μm, for example under 25 μm, for example under 20 μm, for example under 10 μm. More preferably, the compound has a D₅₀ in the range 0.5-50 μm, for example 0.5 to 20 μm, for example 0.5 to 10 μm, for example 1.0 to 10 μm, for example 1.5 to 7.5 μm, for example 2.8 to 5.5 μm. Preferably, the compound has a D₉₀ size of under 100 μm. More preferably, the compound has a D₉₀ size of under 75 μm, for example under 50 μm, for example under 25 μm, for example under 20 μm, for example under 15 μm. The compound preferably has a D₅₀ in the range 5 to 100 μm, for example 5 to 50 μm, for example 5 to 20 μm, for example 7.5 to 15 μm, for example 8.2 to 16.0 μm. Preferably, the compound has a D₁₀ in the range 0.5-1.0 μm. Preferably, the compound of formula (I) or salt thereof (e.g. urolithin A) has a D₅₀ in the range 8.2 to 16.0 μm, a D₅₀ in the range 2.8 to 5.5 μm and a D₁₀ in the range 0.5 to 1.0 μm.

In a further embodiment, the compound of formula (I) or salt thereof has a size distribution selected from one of the following:
(i) D₅₀ size in the range 0.5 to 50 μm and a D₉₀ size in the range 5 to 100 μm,
(ii) the compound has a D₉₀ size in the range 8.2 to 16.0 μm, a D₅₀ size in the range 2.8 to 5.5 μm and a D₁₀ size in the range 0.5 to 1.0 μm;
(iii) the compound of Formula (I) has a D₅₀ size in the range 0.5 to 20 μm and a D₉₀ size in the range 5 to 50 μm;
(iv) the compound of Formula (I) has a D₅₀ size under 50 μm and a D₉₀ size under 75 μm;
(v) the compound of Formula (I) has a D₅₀ size under 25 μm and a D₉₀ size under 50 μm;
(iv) the compound of Formula (I) has a D₅₀ size under 10 μm and a D₉₀ size under 20 μm;
(v) the compound of Formula (I) has a D₅₀ size under 10 μm and a D₉₀ size under 15 μm; or
(vi) the compound of Formula (I) has a D₅₀ size of 10 μm and a D₉₀ size of 20 μm.

In one embodiment, the composition is administered in the form of a composition comprising: a) a medium-chain triglyceride; b) the compound of formula (I) or salt thereof; and one or more retinoid compounds. Within those embodiments, preferably the compound of formula (I) (e.g. urolithin A) is in micronized form. Compositions comprising a urolithin or salt thereof, and a medium chain triglyceride can be found in International patent application: WO2017/036992.

For the avoidance of doubt, compounds in compositions of the invention may be formulated in the same composition or formulated in separate compositions for simultaneous, separate or sequential administration.

### Kits

Also within the scope of the present invention are kits, comprising composition of the invention. Kits typically include a label indicating the intended use of the contents of the kit and instructions for use. The term “label” includes any writing, or recorded material supplied on or with the kit, or which otherwise accompanies the kit.

The invention further provides kits comprising:
(a) a topical formulation comprising
   (i) a compound of formula (I), or a salt, prodrug, metabolite or derivative thereof, as defined herein;
   (ii) one or more retinoid compounds;
(b) a container, or containers, for containing said agents; and
(c) optionally instructions for simultaneous, separate or sequential administration.

In a further embodiment, the invention provides a kit comprising:
(a) an oral composition comprising a compound of formula (I), for example urolithin A for example, about 100 mg to about 2000 mg of a compound of formula (I), for example, urolithin A, or a salt, prodrug, metabolite or derivative thereof;
(b) a topical composition comprising one or more retinoid compounds;
(c) a container, or containers, for containing said compositions; and
(d) optionally instructions for simultaneous, separate or sequential administration.

### Preparation of Compositions of the Invention

In a further embodiment, there is provided a process for preparing a composition of the invention, which comprises, mixing
(a) a compound of formula (I), or a salt, prodrug, metabolite or derivative thereof, for example, urolithin A;
(b) one or more retinoid compounds, and
(c) one or more excipients, for example, one of more excipients suitable for topical administration.

The term ‘about’ refers to a tolerance of ±20% of the relevant value, for example ±15% of the relevant value, such as ±10% of the relevant value or ±5% of the relevant value.

The term ‘excipient’ refers to a substance formulated alongside the active ingredient of a medication, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing

The term “pharmaceutically acceptable” means approved by a regulatory agency of the Federal or a state government or listed in the U.S. Pharmacopeia or other generally recognized pharmacopeia for use in animals, and more particularly in humans.

The term ‘retinoid’ refers to a group of compounds that are natural derivatives of vitamin A or are chemically related to it. Retinoids are grouped by their ‘generation’ based on their molecular structure and receptor selectivity, and the term ‘retinoids’ includes all such retinoids, for example:
First generation retinoids, including retinol, retinal, tretinoin (retinoic acid), isotretinoin, and alitretinoin;
Second generation retinoids, including etretinate and its metabolite acitretin;
Third generation retinoids, including adapalene, bexarotene, and tazarotene; and
Fourth generation retinoids, including trifarotene.

The term, “separate” administration means the administration of each of two or more compounds to a patient from non-fixed dose dosage forms simultaneously, substantially concurrently, or sequentially in any order. There may, or may not, be a specified time interval for administration of each the compounds.

The term “sequential” administration means the administration of each of two or more compounds to a patient from non-fixed (separate) dosage forms in separate actions. The administration actions may, or may not, be linked by a specified time interval. For example, administering compounds over a specified time such as once every 14 to 21 days.

The term “simultaneous” administration means the administration of each of two or more compounds to a patient in a single action such as where each compound are administered independently at substantially the same time or separately within time intervals that allow the compounds to show' a cooperative therapeutic effect.

The term “therapeutically effective amount” as used herein refers to the amount of a compound or compounds that, when administered, is sufficient to prevent the development of, or to relieve to some degree, one or more symptoms of the disease that it targets. The particular dose of each compound administered according to this invention will of course be determined by the particular conditions surrounding the case, including the compound administered, the route of administration, the particular condition being treated, as well as considerations such as age, weight and sex of the treated subject.

The invention will now be illustrated with respect to the following non-limiting examples

### EXAMPLES

The invention will now be illustrated with respect to the following non-limiting examples

The anti-aging and anti-photoaging effects of retinoids are based on the promotion of collagen fibre synthesis, and the inhibition of collagen degradation by inhibiting metalloproteinase (MMP) activity. For instance, retinoic acid at 1 uM mildly reduced MMP1 and increases pro-collagen formation in UVA-irradiated human dermal fibroblasts (Oh et al (2020) Int J Mol Sci 21(20), 7756). A more robust increase in pro-collagen was observed with higher doses of retinoic acid (20 uM) (PMID: 15009115). However, high doses of retinoids have been associated with side effects including photosensitivity and local irritation. For instance, 10 uM retinol treatment induced the pro-inflammatory cytokines interleukin-8 (IL-8) and MCP-1 in human dermal fibroblasts, keratinocytes and melanocytes (PMID: 14615068).

We therefore investigated the ability of Urolithin A to:
Prevent side effects of higher doses or retinol, maintaining its collagen synthesis ability
Boost the collagen producing ability of retinol at lower doses, doses that are not inducing inflammation but are suboptimal for collagen synthesis

### Example 1. Urolithin A Boosts Reduction of MMP1 Gene Expression Induced by Retinoic Acid in Human Dermal Fibroblasts (HDF)

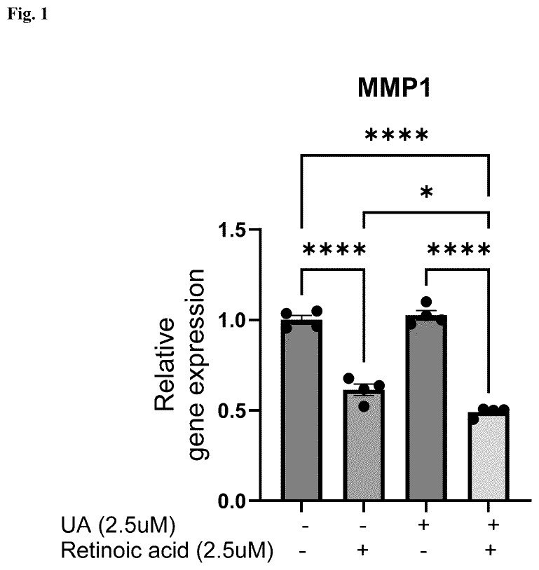

In one experiment, HDF cells were exposed to certain doses of retinoic acid (2.5 uM, 10 uM), of urolithin A (UA) (2.5 uM, 10 uM), combinations of retinoic acid and UA, and DMSO (as control) to assess the impact on matrix metalloproteinase (MMP) expression. Treatments were applied to HDF for 24 h. After 24 h, experiment was stopped, RNA extracted and gene expression of MMP1 was measured by qPCR. Consistently with literature (Oh et al (2020) Int J Mol Sci 21(20):7756), retinoic acid reduces MMP1 expression in HDF at low dose of 2.5 uM by 51% (FIG. 1 and Table 1). Addition of UA at same low dose did not significantly impact MMP1 expression. Surprisingly, combining UA and retinoic acid at 2.5 uM results in a synergistic and statistically significant decrease in MMP1 expression. The data in FIG. 1 indicate that retinoic acid plus UA is significantly more effective that retinoic acid alone. In particular, the combination of compounds reduced MMP1 level by a further 20% compared to retinoic acid alone. It also indicates that low doses of UA, that are not effective on MMP1 expression, show surprising activity when combined with retinoic acid.

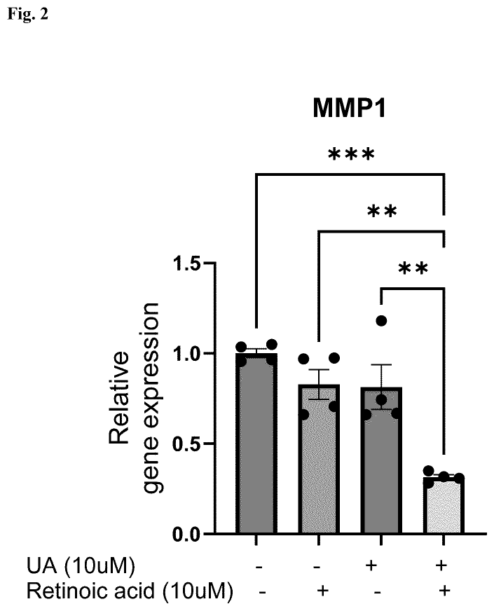

We also tested two higher doses (10 uM) of UA, retinoic acid or their combinations on MMP1 expression (FIG. 2 and Table 2). Retinoic acid at this 10 uM dose is less effective in reducing MMP1. This mild non-significant results might be due to side effects/toxicity induced at this higher dose. UA at 10 uM showed a similar mild non significant reduction on MMP1 expression. However, combining UA and retinoic acid at 10 uM results in a synergistic and statistically significant decrease in MMP1 expression. The data in FIG. 2 therefore indicate that, at these doses, retinoic acid plus UA is significantly more effective by 61-62% compared to retinoic acid alone or UA alone.

### Table 1

| | Change in | | |
|---|---|---|---|
| | MMP1 | | Adjusted |
| Tukey's multiple comparisons test | expression | Summary | P Value |
| DMSO vs. UA 2.5 uM | 2% | Ns | 0.9004 |
| DMSO vs. R. 2.5 uM | −39% | **** | <0.0001 |
| UA 2.5 uM vs. UA 2.5 uM + R. 2.5 uM | −51% | **** | <0.0001 |
| R. 2.5 uM vs. UA 2.5 uM + R. 2.5 uM | −20% | * | 0.0215 |
| DMSO vs. UA 2.5 uM + R. 2.5 uM | −51% | **** | <0.0001 |

### Table 2

| | Change in | | |
|---|---|---|---|
| | MMP1 | | Adjusted |
| Tukey's multiple comparisons test | expression | Summary | P Value |
| DMSO vs. UA 10 uM | −19% | ns | 0.3442 |
| DMSO vs. R. 10 uM | −17% | ns | 0.408 |
| UA 10 uM vs. UA 10 uM + R. 10 uM | −61% | ** | 0.0028 |
| R. 10 uM vs. UA 10 uM + R. 10 uM | −62% | * | 0.0215 |
| DMSO vs. UA 10 uM + R. 10 uM | −69% | ** | 0.0022 |

### Example 2. Urolithin A and Retinol Synergistically Increase PINK1 Expression in Human Primary Keratinocytes (NHEK)

Human primary keratinocytes (NHEK) were treated for 24 hours with either Urolithin A (UA) alone, retinol alone, or a combination of both compounds (each at 10 μM). PINK1 expression was measured as a marker of mitochondrial quality control.

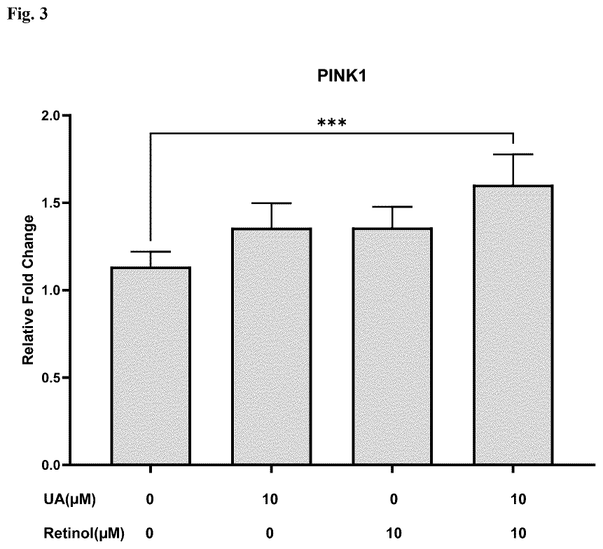

Urolithin A alone induced an increase in PINK1 expression (+35.81%, FIG. 3), consistent with its known role in promoting mitophagy. Retinol alone also showed a similar an increase of PINK1 expression (+35.94%, FIG. 3), which is novel and unexpected, as retinol is primarily associated with skin renewal rather than mitochondrial signaling.

Interestingly, the combination of Urolithin A and retinol resulted in a statistically significant effect, with PINK1 expression increasing dramatically by +60.43% compared to untreated control (***p<0.001, FIG. 3). This dramatic enhancement indicates that co-treatment may activate mitochondrial stress response pathways more effectively than either compound alone.

A 60% increase is both dramatic in magnitude, significant, translationally relevant and unexpected with the use of small molecules such as UA and retinol. This effect is indeed comparable to intense and gold standard mitophagy activating interventions such as exercise and electric stimulation (Li et al (2021) Biomedicines 9, 701, FIG. 3D). These effects are striking also considering that experiments are performed in healthy cells with fully functional mitophagy machinery. As reference, increases of 60% or more in PINK1 levels can be observed only in conditions where cells have impaired mitophagy and with the use of pharmacological agents such as the well known autophagy/mitophagy inducer metformin (Xie et al (2025) Int J Biol Sci 21(8):3505-3526, FIG. 6C).

PINK1 is a key marker of mitochondrial quality control and stress response. Mitochondrial dysfunction contributes to skin barrier impairment and irritation. The observed increase in PINK1 with Urolithin A and retinol indicates enhanced mitochondrial resilience, which may help reduce irritation, a common side effect of retinol.

Table 3: Comparison of percentage changes in PINK1 mRNA expression across treatment groups. Percent increase in PINK1 expression was calculated relative to the indicated comparator (control). Statistical significance was determined by one-way ANOVA. ns: not significant; ***p<0.001.

| | % increase in PINK1 | Statistical |
|---|---|---|
| Comparison | expression | Significance |
| Urolithin A 10 μM vs Control | 35.81 | |
| Retinol 10 μM vs Control | 35.94 | |
| Urolithin A 10 μM + Retinol 10 μM vs | 60.43 | *** |
| Control | | |

### Example 3. Urolithin A Protects Against Skin Irritation Markers Induced by Retinol in Human Keratinocytes

Interleukin-1 alpha (IL1α) is a key pro-inflammatory cytokine involved in the skin's response to external stressors and irritants. Its expression is rapidly upregulated in keratinocytes following exposure to irritants such as retinol, making it a reliable biomarker for assessing skin irritation and inflammation. Elevated IL1α levels are associated with compromised skin barrier function and increased sensitivity, which are common side effects of retinol-based treatments. Therefore, we investigated IL1α expression as a translationally relevant and quantifiable measure of irritation. In this study, the ability of Urolithin A to suppress IL1α expression highlights its potential as a protective agent to improve the tolerability of retinol-containing formulations.

Human primary keratinocyte cells were treated with 10 μM retinol for 24 hours to induce irritation, and inflammation was assessed by quantifying the expression of the cytokine interleukin-1 alpha (IL1α). To evaluate the protective effect of Urolithin A, cells were co-treated with retinol and either 2.5 μM or 10 μM Urolithin A. IL1α expression was measured and compared across treatment groups.

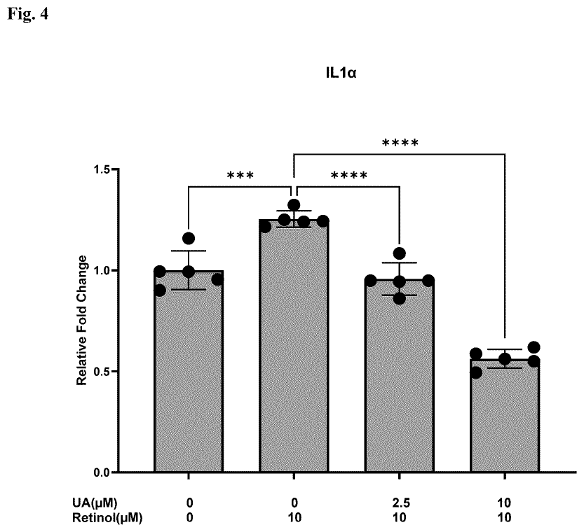

Retinol alone significantly increased IL a expression by +25.43% compared to untreated control cells (p<0.001), confirming its pro-inflammatory effect. Co-treatment with 2.5 μM Urolithin A resulted in a −23.67% reduction in IL1α expression compared to retinol alone (p<0.0001), although the change relative to control was not statistically significant (−4.26%, ns). Notably, co-treatment with 10 μM Urolithin A led to a −55.14% reduction in IL1α expression compared to retinol alone (p<0.0001), and a −43.74% decrease compared to control (p<0.0001), indicating a strong anti-inflammatory effect (FIG. 4 and Table 4).

These results demonstrate that Urolithin A significantly mitigates retinol-induced irritation in a dose-dependent manner, with 10 μM showing robust protective activity.

Table 4: Comparison of percentage changes in IL1α mRNA expression across treatment groups. Percent increase or decrease in IL1α expression was calculated relative to the indicated comparator (control or retinol 10 μM). Statistical significance was determined by one-way ANOVA. ns: not significant; ***p<0.001; ****p<0.0001.

| | % increase or | |
|---|---|---|
| | decrease in IL1a | Statistical |
| Comparison | expression | Significance |
| Retinol 10 μM vs Control | +25.43 | *** |
| Urolithin A 2.5 μM + Retinol 10 μM vs | −23.67 | **** |
| Retinol 10 μM | | |
| Urolithin A 10 μM + Retinol 10 μM vs | −55.14 | *** |
| Retinol 10 μM | | |

### Methods

### Cell Culture and Treatments

Normal human epidermal keratinocytes (NHEK; PromoCell, C-12003) were expanded as monolayers in T-75 cm² tissue culture flasks using Keratinocyte Growth Medium 2 (KGM2; PromoCell, C-20011), a serum-free medium supplemented to final concentrations of bovine pituitary extract 0.004 ml/ml, recombinant human epidermal growth factor 0.125 ng/ml, recombinant human insulin 5 μg/ml, hydrocortisone 0.33 pg/ml, epinephrine 0.39 pg/ml, recombinant human transferrin 10 μg/ml, and CaCl₂ 0.06 mM, with 1% penicillin-streptomycin (Biowest, L0022-100). Cultures were maintained at 37° C. in a humidified incubator with 5% CO₂, the growth medium was renewed every other day, and cells were detached at confluence using trypsin-EDTA and subcultured according to standard procedures.

Urolithin A (Amazentis SA) was prepared in DMSO to a 50 uM stock solution and diluted in medium to the indicated working concentrations. Retinol (MedChemExpress, CAS 302-79-4) was supplied as a 10 mM stock solution in DMSO and was used at the specified doses.

### Cell Seeding, Treatment

To evaluate changes in PINK1, IL1α gene expression, a single-cell suspension of NHEK was prepared and 75,000 cells were seeded per well in 24-well plates, followed by a 24-hour incubation to allow attachment and recovery. After this 24-hour period, cells were treated for 24 hours with retinol (MedChemExpress, CAS 302-79-4) at 10 μM, or with combinations comprising retinol at 10 μM plus urolithin A at 2.5 μM or 10 μM; vehicle was matched across conditions and the final DMSO concentration was held constant. At the conclusion of the 24-hour treatment, samples were collected, and total RNA was isolated.

RNA Isolation and qPCR

Total RNA was isolated from normal human epidermal keratinocytes (NHEK) using TRIzol reagent (Thermo Fisher Scientific, Cat. No. 15596026) according to the manufacturer's instructions. Complementary DNA (cDNA) was synthesized from total RNA using the QuantiTect Reverse Transcription Kit (Qiagen, Cat. No. 205313) following the manufacturer's protocol.

Quantitative real-time PCR (qPCR) was performed on a QuantStudio 6 Flex Real-Time PCR System (Life Technologies) using either probe-based or dye-based detection chemistries in accordance with standard TaqMan and SYBR Green methodologies for real-time quantification and cycle threshold (Ct) analysis. Probe-based reactions utilized TaqPath ProAmp Master Mix (Applied Biosystems, Thermo Fisher Scientific, Cat. No. A30866), while dye-based reactions employed PowerUp SYBR Green Master Mix (Applied Biosystems, Cat. No. A25742), both used per respective manufacturer recommendations.

TaqMan assays used the following probes: GAPDH (Hs99999905_m1), RPLP2 (Hs01115128_gH), and PINK1 (Hs00260868_m1). SYBR Green assays were conducted with the following primer sets:

IL1A
forward
5′-TGTATGTGACTGCCCAAGATGAAG-3′
and
reverse
5′-TGTATGTGACTGCCCAAGATGAAG-3′;
YWHAZ
forward
5′-CCTGCATGAAGTCTGTAACTGAG-3′
and
reverse
5′-GACCTACGGGCTCCTACAACA-3′;
RPLP2
forward
5′-TCTTGGACAGCGTGGGTATCGA-3′
and
reverse
5′-CAGCAGGTACACTGGCAAGCTT-3′.

Quantitative PCR results were normalized to the mean expression of housekeeping genes GAPDH and RPLP2 and analyzed using the comparative Ct (ΔΔCt) method. All qPCR operations were conducted on the QuantStudio 6 Flex platform using appropriate settings for probe or dye chemistry. Statistical analyses of gene expression changes were performed using one-way ANOVA.

### Example 4. Urolithin A Boosts Collagen Production Induced by Retinoic Acid at Suboptimal Doses in Human Dermal Fibroblasts (HDF)

Human dermal fibroblasts (HDF cells) are exposed to certain doses or retinoic acid (0.1 uM, 0.5 uM, 1 uM, 2.5 uM, 5 uM) to assess the impact on collagen synthesis. Three different doses of UA as above (or DMSO as control) are used to investigate UA's ability to enhance retinol-mediated collagen production, that at these doses is low or unchanged. As above, UA doses or DMSO are applied either 24 h before or at the same time as retinoic acid exposure. After 24 h, experiment are stopped, RNA extracted and gene expression of collagen genes and collagen degrading enzymes was measured by qPCR.

### Example 5. Urolithin A and Retinoic Acid have Synergistic Effects in Mitophagy Activation in Human Dermal Fibroblasts (HDF)

Human dermal fibroblasts (HDF cells) are exposed to (i) retinoic acid (0.1 uM, 0.5 uM, 1 uM, 2.5 uM, 5 uM); UA (0.5, 2.5, 10 uM), or (iii) combinations of both UA and retinoic acid. Treatment duration for 24 or 48 hours. Cells are used to analyse biomarkers associated with increased mitophagy by qPCR.

### Example 6. Urolithin A and Retinoic Acid have Synergistic Effects in Activation of Mitochondrial Respiration in Human Dermal Fibroblasts (HDF)

Human dermal fibroblasts (HDF cells) are exposed to (i) retinoic acid (0.1 uM, 0.5 uM, 1 uM, 2.5 uM, 5 uM); UA (0.5, 2.5, 10 uM), or (iii) combinations of both UA and retinoic acid. Treatment duration for 24 or 48 hours. Cells are used to measure mitochondrial respiration by Seahorse respirometer.

### Example 7. Urolithin A Protects Against Inflammation Induced by Retinoic Acid in Human Dermal Fibroblasts (HDF)

We investigate the protective effects of urolithin A against cellular dysfunction induced by retinoic acid in human dermal fibroblasts (HDF cells). In a first experiment, different doses of retinoic acid are applied for 24 h to human dermal fibroblasts (HDF cells) to induce inflammation, as described in the method section below and in Kim et al (2003) Toxicol Lett. 146(1):65-73 (PMID: 14615068). The doses inducing inflammation are selected for a second experiment. Here, cells are pre-treated with urolithin A at 3 different doses (0.5 uM, 2.5 uM, 10 uM) or DMSO as control for 24 h. Then cells are exposed to the previously selected doses of retinoic acid. After 24 h the release of pro-inflammatory cytokines (IL-6, IL-8, IL-11, MCP-1) is measured in cell culture media to assess the ability of UA to rescue inflammation induced by retinoic acid

In a separate experiments exposure of UA or DMSO (as control) is concomitant to the exposure to retinoic acid. After 24 h the release of pro-inflammatory cytokines is measured in cell culture media to assess the ability of UA to rescue inflammation induced by retinoic acid.

### Methods

### Cell Culture

Study was conducted on Normal Adult Human Dermal Fibroblasts (NHDF-Ad) (Promocell). The primary skin cells were amplified in flask and seeded in 12-well cell culture plates, filled with 1 mL of culture medium and then incubated at 37° C. with 5% CO₂ and saturated hygrometry. The basic culture medium of human dermal fibroblasts was composed Fibroblast Growth Medium 2+growth Factors (Promocell, C-23220 and C-39325). Cells were seeded in 6 well plate at a confluence of 7000 cell/cm2. The following day cells were treated with (i) retinoic acid (MedChem Express, HY-14649) or Urolithin A (Amazentis) at different concentrations as indicated in the examples.

### ELISA

24 hours after treatment, the medium was collected and an ELISA for Interleukin-6, Interleukin-8 and MCP-1 were performed following the instruction provided by the supplier (Proteintech ref. KE10007). Finally, the absorbance at a wavelength of 450 nm was measured using a microplate plate reader (Fluostar Optima, BMG Labtech).

gPCR

RNA from HGF cells was extracted using TRIzol (Thermo Scientific, 15596026) and then transcribed to cDNA by the QuantiTect Reverse Transcription Kit (Qiagen, 205313) following the manufacturer's instructions. The q-RT-PCR reactions were performed using the TaqPath ProAmp master mix (Applied biosystems, A30866) and the expression of selected genes listed below was analysed using the Quantstudio 6 flex (Life technologies) and following TaqMan probes listed. All quantitative polymerase chain reaction (PCR) results were presented relative to the mean of housekeeping genes (ΔΔCt method). TaqMan probes: GAPDH: Hs99999905_m1; RPLP2 Hs01115128_Gh; LC3: Hs00797944_s1; P62/SQSTM1: Hs00177654_m1; PINK1: Hs00260868_m1, COL1A1: HS00164004_m1; IL1b: Hs00174097_m1; IL6: Hs00174131_m1; MMP1: Hs00899658_m1; MMP3: Hs00968305_m1. The analysis was performed with Quantstudio 6 Flex (Life Technologies). All quantitative polymerase chain reaction (qPCR) results were presented relative to the mean of housekeeping genes (ΔΔCt method). mRNA levels were normalized over the average GAPDH and RPLP2. Statistical analysis: one-way ANOVA.

### Seahorse

Oxygen consumption was measured using the XF96 Extracellular Flux Analyzer (Agilent). Once reaching the appropriate confluence, HGF cells shifted in DMEM-based substrate limited medium, containing 0.5 mM glucose, 1 mM GlutaMAX, 0.5 mM carnitine, and treated with either DMSO (as control), AgNPs, F, or UA. 45 min before the assay, cells were washed two times with Assay Medium (111 mM NaCl, 4.7 mM KCl, 1.25 mM CaCl2, 2 mM MgSO4, 1.2 mM NaH2PO4, 2.5 mM glucose, 0.5 mM carnitine, oleic acid 5% and 5 mM HEPES adjusted to pH 7.4 at 37° C. on the day of the assay). Cells were then incubated for 30 min in a non-CO2 incubator. Oxygen consumption rate was determined at maximal level, after the addition of FCCP.

### EQUIVALENTS

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

1. A composition comprising:
(a) a compound of formula (I), or a salt, prodrug, or metabolite thereof; 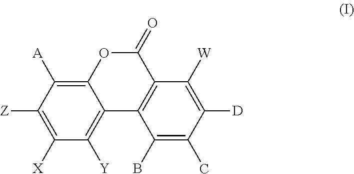
wherein:
A, B, C, D, W, X, Y and Z are each independently selected from H and OH; and
(b) one or more retinoid compounds.

2. The composition as claimed in claim 1, wherein the retinoid compound is a first-generation retinoid, for example, a retinoid compound selected from one or more of retinol (and esters thereof), retinal, tretinoin (all trans retinoic acid), isotretinoin (13 cis-retinoic acid), and alitretinoin (9 cis-retinoic acid).

3. The composition as claimed in claim 2, wherein the retinol ester is selected from one or more of retinol acetate, retinol palmitate, and retinol propionate.

4. The composition as claimed in claim 1, wherein the retinoid compound is a second-generation retinoid, for example, a retinoid compound selected from one or more of etretinate and acitretin.

5. The composition as claimed in claim 1, wherein the retinoid compound is a third-generation retinoid, for example, a retinoid compound selected from one or more of adapalene, bexarotene, etarotene, tazarotene, motretinide, arotinoide and arotinoidethylester.

6. The composition as claimed in claim 1, wherein the retinoid compound is a fourth-generation retinoid, for example, trifarotene.

7. The composition as claimed in claim 1, wherein the retinoid compound is tazarotene.

8. The composition as claimed in claim 1, wherein the retinoid compound is selected from Seletinoid G, AGN 193109, AGN-190168, AGN-190299 and Rexinoid analogues such as, LGD1069 and LG100268.

9. The composition as claimed in claim 1, wherein the compound of formula (I) is selected from one or more of urolithin A, urolithin B, urolithin C and urolithin D.

10. The composition as claimed in claim 1, wherein the compound of formula (I) is urolithin A.

11. (canceled)

12. A method of treating or preventing a skin disease, disorder, or condition, comprising administering to a subject in need thereof an effective amount of the composition as claimed in claim 1.

13. The method as claimed in claim 12, wherein the skin disease, disorder or condition is selected from acne, dermatitis, eczema, hives, liver spots (lentigo), lupus erythematosus, hyperpigmentation, keratosis pilaris, melasma, psoriasis, rosacea, shingles or warts, for example, wherein the acne is selected from acne vulgaris, nodulocystic acne, and acne conglobata.

14. The method as claimed in claim 12, wherein the skin disease, disorder or condition is selected from skin infection, skin irritation, skin inflammation or skin damage caused by radiation, for example, UV, beta or gamma radiation, for example, during medical treatment for a condition, such as a cancer, such as cutaneous T-cell lymphoma, and Kaposi's sarcoma.

15. A non-therapeutic method for enhancing skin health, comprising administering to a generally healthy subject in need thereof an effective amount of the composition as claimed in claim 1.

16. A non-therapeutic method for reducing, slowing or preventing skin aging, and/or lowering skin biological age, comprising administering to a subject in need thereof an effective amount of a composition as claimed in claim 1.

17. A non-therapeutic method for reducing or preventing skin wrinkles and/or maintaining or improving skin barrier function, comprising administering to a subject in need thereof an effective amount of a composition as claimed in claim 1.

18. A non-therapeutic method of:
a) maintaining or improving skin hydration and/or skin elasticity;
b) reducing irradiation-induced skin damage, for example, damage caused by UV radiation;
c) improving or boosting skin collagen levels;
d) supporting collagen function and/or architecture;
e) preventing collagen decline;
f) supporting skin collagen levels; and/or
g) maintaining or enhancing skin collagen health,
comprising administering to a subject in need thereof an effective amount of a composition as claimed in claim 1.

19. A method for reducing adverse effects of the administration of a retinoid compound, comprising administering to a subject in need thereof an effective amount of a composition as claimed in claim 1.

20. (canceled)

21. The composition as claimed in claim 1, comprising:
(a) 0.1% (w/w) to 5% (w/w) of the compound of formula (I), or a salt, prodrug, or metabolite thereof; and
(b) 0.1% (w/w) to 1% (w/w) of one or more retinoid compounds,
wherein the composition is formulated for topical administration.

22. The composition as claimed in claim 21, wherein the composition is formulated as a cream.
